(19) United States
(12) Patent Application Publication
(10) Pub. No.: US 2003/0215484 A1
(43) Pub. Date: Nov. 20, 2003

(54) Ceramic surface layers and coated devices

(76) Inventors: Niklas Axen, Jarlasa (SE); Kajsa Bjorklund, Uppsala (SE); Leif Hermansson, Uppsala (SE)

Correspondence Address: YOUNG & THOMPSON, 745 SOUTH 23RD STREET 2ND FLOOR, ARLINGTON, VA, 22202

(21) Appl. No.: 10/377,723

(22) Filed: Mar. 4, 2003

(30) Foreign Application Priority Data
Mar. 4, 2002 (SE) 0200637-7

(51) Int. Cl. A61F 2/02; A61L 2/00; B05D 3/00

(52) U.S. Cl. 424/423; 427/2.27; 623/11.11

(57) ABSTRACT

Surface coating method for applying a chemically bonded ceramic coating on a substrate, comprising the steps of preparing a powder mixture based on a hydraulic ceramic binder phase, preparing a substrate surface, applying at least one layer of the powder mixture on the substrate, and finally hydrating the powder layer/layers by addition of a water-based solution. The present invention method can be applied without using elevated temperatures, complicated and complex equipment, while maintaining control over the microstructure of the coating. The inventive procedure can suitably be used for producing general orthopaedic and dental implants. The inventive coatings can also be used in microstructure technology applications or in wear and friction applications.

### THE FIELD OF THE INVENTION

The present invention relates to a method of covering devices in part or completely, with ceramic layers or coatings composed of specific ceramic materials based on hydraulic binders. In particular, the present invention relates to a method of coating medical devices for implantation with coatings of biocompatible ceramics.

### BACKGROUND OF THE INVENTION

Ceramics

Ceramic materials are generally hard and chemically stable. Ceramics also tend to be brittle and difficult to machine to desired shape. Many ceramics also exhibit minimal reaction with organic tissues, and are therefore suitable for implantation.

In orthopaedics, implants made from hydroxyapatites or other calcium phosphate ceramics are of particular interest. Hydroxyapatite is uniquely osseo compatible, since bone tissue regenerates excellently in contact with this ceramic. The material seems to be capable of forming a direct bond with natural bone. Orthopaedic implants with coatings of hydroxyapatite deposited by using Thermal Spray Deposition (TSD), are commercially explored. Also calcium carbonates are explored for orthopaedic applications. Examples are the bone graft materials: Norian SRS®, Osteoset® and Biobon®.

There are many types of calcium phosphates. A split into six typical types is sometimes made: Dicalciumphosphate dihydrate, anhydrous dicalciumphosphate, octacalciumphosphate, beta-tricalciumphosphate, hydroxyapatite and tetracalciumphosphate monoxide. The latter three are of specific interest for ortopaedic applications.

As pure bulk material hydroxyapatites and other calcium phosphates have poor mechanical properties. Hydroxyapatite is therefore often used as coatings supported on metal substrates or as an additive in a stronger matrix. Polymer based bone cements with hydroxyapatite fillers are established p roducts. However, all techniques involving elevated temperatures tend to alter the microstructure of the hydroxyapatite, e.g. the hydration water in the hydroxyapatites may leave the structure.

For some ceramic materials, the hardening and densification processes follow as a result of chemical reactions between ceramic powders and water, i.e. hydration. This group of materials, which is referred to as hydraulic cements, ranges from concrete based on Portland cement to special ceramics used in dentistry and orthopaedics. Hydraulic cements include for example sulphates, silicates, aluminates and carbonates of calcium. A tooth filling material based on calcium aluminate is described in: PCT/SE99/01803, “Dimension stable binding agent systems”, filed in Oct. 8, 1999.

Binding phase systems based on hydrated calcium aluminate have unique properties. In comparison to other water binding systems, for example silicates, carbonates and sulphates of calcium, the aluminates are characterised by high chemical resistance, high strength and a relatively rapid hardening.

The high strength of calcium aluminate cements is due to the high absorption capacity of hydration water, which in turn results in a low residual water content and low porosity. The high compaction also increases the resistance to corrosion.

Most ceramics components are manufactured with techniques involving preparation of ceramic powders, realisation of green-bodies of said powders and densification by sintering at high temperatures and/or pressures. By applying the heat/pressure, pre-shaped green-bodies are compacted to dense and strong components.

Traditionally, cement processing involves preparation of the raw material by high temperature processing of selected minerals, grinding of the raw materials to fine powders, mixing of powder and water, possibly together with additives controlling strength, rheology and hardening rate, subsequent shaping of the mixture, and final hardening/solidification by hydration reactions. It is known within the field that improved mechanical strength of hydraulic cements is achieved if the cement powder is compacted by mechanical means, prior to the hydration. The compaction may be applied to the dry cement powder, or to the powder-liquid paste. The most probable explanation for the better strength hence obtained, is that bodies of higher degrees of compaction and less porosity are obtained.

For the more established applications of cements, e.g. concrete based on Portland cement, manufacturing procedures involving pre-compaction and subsequent hydration are however often unsuitable due to the degree of complexity of the methods involving compaction of the powders using high pressures. In special application s, more complicated processing techniques may however be worthwhile.

Surface Coating Techniques

Ceramics are also exploited as thin layers and surface coatings. Coating technology offers a means for combining the properties of substrate materials, such as strength, ductility, low weight or machinability, with the different properties of surface layers of other materials, e.g. hardness, wear resistance, esthetical aspects, chemical resistance, or biocompatibility.

A range of surface coating techniques are established. The most established techniques for applying of ceramic coatings are Chemical Vapor Deposition (CVD), Physical Vapor Deposition (PVD), Thermal Spray Deposition (TSD) and Electrolytic Deposition (ED). Coatings may also be produced with powder technology.

A great disadvantage with these techniques for deposition of ceramic films, except for ED, is the elevated temperatures involved in the processes. This sets limitations to the selection of substrate materials, and to the chemical structures and phases that can be achieved in the coating. Another disadvantage may be the complexity of the required equipment, involving gas-tight vacuum arrangements for CVD and PVD, and high-temperature presses for powder technology.

TSD involves very high cooling rates for the deposition material; from typically 10 000° C. to ambient temperature within a few microseconds. Under such conditions, accurate control of the microstructure of the coating material is not possible. Nor can phase-structure, chemical composition, porosity or surface structure be regulated, due to the temperatures required in the process.

Throughout this application the term biocompatibility is used a number of times implying certain properties on the material or surface in question. However, it should be noted that biocompatibility is used as a generic term for the different properties that are required or desirable for materials that are to be in contact with biological tissue. Moreover, the material has to be used/prepared in the right way and to be used in suitable applications.

Another frequently used term is osseo-compatible, which implies that a material is especially advantageous for use in contact with bone tissue. As presented above, some osseo-compatible materials even seem to be capable of forming a direct bond with natural bone. Examples of materials considered to be osseo-compatible are hydroxyapatite, and coral-like materials of calcium carbonates.

### SUMMARY OF THE INVENTION

In view of the drawbacks associated with the prior art methods for achieving surface coatings and coating materials, there is a need for uncomplicated, low-temperature techniques for generating biocompatible and mechanically strong surface coatings of hydraulic ceramics.

Therefore, the object of the present invention is to provide a method for preparing coatings of chemically bonded hydraulic cements without using elevated temperatures, without complicated and sophisticated equipment, while maintaining control over the microstructure of the coating.

The present invention achieves this object with the method defined in claim 1, and the coatings defined in claims 25-30.

One advantage with the present invention is that the powder containing hydraulic cement is applied to the pretreated substrate surface without initiation of the hydration process, whereby the rate of hydration does not have to be taken into account. This is very advantageous when comparing with the case when water slurries are sprayed onto a substrate. In the case of obtaining ceramic coatings using water slurries, the rate of hydration is difficult to adjust, since a slow hardening is desired for the flexibility of the spraying process but a rapid hardening is preferred after deposition to prevent the layer from moving.

Another advantage of the inventive procedure is the possibility to manipulate the powder layer prior to hardening, e.g. compaction of the powder by pressure, partial removal of layer, replacement of layer, application of secondary layers, or creation of microstructures in said layers, etc.

Yet another advantage of the inventive procedure is that it allows selecting high temperature deposition techniques for the powder, such as TSD, since the non-hydrated ceramic is capable of withstanding the temperatures emplyed in such techniques, whereas the hydrated ceramic does not.

The inventive procedure also provides for the control of the hydration conditions, for example determining the starting time of the hydration. The surrounding temperature, and the delivery technique for the water (e.g. as vapour or liquid) may be selected with more flexibility if the start of the hydration can be controlled.

The biocompatible surface coatings obtainable with the inventive procedure can suitably be used for producing general orthopaedic and dental implants. The inventive coatings can also be used in microstructure technology, for the fabrication of controlled surface structures, or in wear and friction applications.

### DETAILED DESCRIPTION OF THE INVENTION

The present invention relates to a method for generating coatings of chemically bonded hydraulic ceramics. With the invention, biocompatible surface coatings suitable for general orthopaedic and dental implants can be generated. The inventive coatings can also be applied in microstructure technology and wear and friction applications.

The application of coatings of hydrating cements is treated in the pending patent application SE-0 104 440-3 with the title “Coating method and coating devices”, which describes the application of slurries of certain hydraulic cements and water solutions, to pretreated substrates.

In the present invention, the application method is different, and the properties of the coatings are improved.

With the method according to the present invention, powder containing hydraulic cement is applied to the pretreated substrate surface without initiation of the hydration process. Thereafter, the powder layer may, if desired, be compacted, and as a final step the hydration is initiated to harden the ceramic layer.

The steps of the inventive method are described in detail below:

A first step comprises selection of a powder from the group of hydraulic cements, optional pretreatment of the powder, primarily powder milling to desired grain size (preferably, such that the grain size is in the range 0.5-20 microns to more than 50 vol.%, preferably to more than 80 vol.% and most preferably more than 90 vol.%), if necessary, burning of the powder to remove any residual water and organic contamination (normally necessary due to contamination of the powder originating from processing, e.g. powder mixing, grain size reduction, etc.), optional addition of ceramic and/or organic additives/fillers. The purpose of the additives may be to provide expansion control, hardness, strength, biocompatibility, or desired rheological properties.

Particularly important hydraulic cements, which act as binder phases, are calcium aluminates and calcium silicates, but also calcium sulphate or calcium carbonate and other similar ceramics are applicable to the invention. Powder compositions of particular interest for the invention are those providing dimension stability, such as the compositions described in PCT/SE99/01803, and those providing a particularly high degree of biocompatibility described in the pending patent application SE-0 104 441-1 with the title “Ceramic material and process for manufacturing”. In a basic form of the present invention, the mixture of ceramic powder only comprises calcium aluminate. A number of stoichiometries exist for the system. Commercially available powders consist mainly of CA or CA₂, where C stands for CaO and A for Al₂O₃, according to accepted cement chemistry notations. The phases C₁₂A₇ and CA₆ and C₃A have also been described in the literature. All phases are applicable on the present invention. Such powders with sufficient quality are commercially available products, e.g. Secar and Ternal White from LaFarge Aluminates.

Generally, if a calcium aluminate powder is mixed with a water-based solution, a hardening process is initiated through a chemical reaction between the calcium aluminate particles and water. More precisely, this hardening process is a hydration, whereby a new binder phase comprised of calcium aluminate hydrate is formed. The hydrates are formed by nucleation of crystalline hydrate phase from the liquid phase. The hydrate is thereafter transformed into different crystalline phases, with a rate depending on e.g. temperature and additives. At room temperature the initially formed hydrate phase is CAH₁₀ where H=H₂O, and the most stable phase is C₃AH₆. Also calcium silicates harden through the formation of hydrates according to a similar route.

As disclosed in our co-pending Swedish patent application SE-0 104 441-1 with the title “Ceramic material and process for manufacturing”, the coating may further comprise a filler material, e.g. for reducing the aluminum content in coating. As it is proposed in said application, calcium titanates, CaTiO₃, or other variants where Ti may be substituted by Zr or Hf and Ca by Mg, Ca, Sr or Ba, in a perovskitic structure, are preferred for this purpose, because they are biologically suitable and they do not substantially affect the mechanical properties of the material. In fact, all material compositions disclosed in said application apply as coating materials in the present invention.

When needed, the ceramic powder is treated by a suitable milling process to obtain a uniform and well-controlled size distribution. One such type of milling process is presented in the example below, but other milling processes known in the field of ceramics could be used as long as the desired result is reached.

A second step involves pre-treatment of the substrate surface, including cleaning and subjecting the substrate to mechanical treatment to obtain a desired surface roughness and structure with mechanical (sand blasting, grinding) or chemical (etching) means. The purpose of such a surface roughness is to improve the coating-substrate adhesion.

It has been shown that a substrate surface pre-treated by sand blasting (as described in the pending patent application SE-0 104 440-3 with the title “Coating method and coating devices”) generates optimal bonding between the coating and substrate. The blasting may be performed in several steps and is preferably done with hard ceramic particles, generating a surface roughness with surface roughness values in the range of 0.1 to 10.0 μm.

Most preferably a primary blasting is performed as a wet blasting, whereby the resulting surface has been shown to be substantially free from blasting material. This is of great importance as the substrate is to be used in biological applications.

The primary blasting may alternatively be another abrasive process producing the same surface roughness, such as grinding with hard particles or grit.

A secondary blasting can be performed with calcium aluminate particles as blasting medium. The secondary blasting should preferably be performed in such a way that calcium aluminate fragments are embedded into the substrate surface. The purpose of this blasting is to achieve a better anchoring of the coating on the substrate, and to provide seed points for the following hydration of the calcium aluminate. This step may be achieved with dry blasting or other impingement method that produces relatively high particle speeds.

To further enhance the bonding between the substrate and the coating, the substrate may thereafter be pretreated with a water solution containing an accelerator component that accelerates the hardening process of the calcium aluminate. Such accelerator components are well known in the field. Lithium chloride (LiCl) has been shown to be an especially suitable accelerator. The purpose of the pretreatment with salt is to initiate the hydrating process in a controlled way directly on the substrate surface, whereby porosity, cracking etc. is avoided at the coating/substrate interface.

The material to be coated, the substrate, may be a ceramic, metallic or polymeric material. Especially important materials are those accepted for use in the field of medical implants, for example titanium, stainless steels, alumina, zirconia and medical grade plastics.

The third step involves application of one or several surface coatings of the cement powder mixture to the substrate. The application procedure may be performed in several ways:

The powder may be applied to the substrate in the form of a slurry, a powder-liquid mix, with which the substrate is covered by dipping, spraying or similar techniques. Subsequently the liquid, e.g. an alcohol or acetone, is evaporated.

Alternatively, the powder could be sprayed onto the surface by a thermal spraying deposition technique (TSD).

Yet another alternative would be to apply the powder in form of a sheet/tape, made e.g. by tape casting, with subsequent evaporation or burning of the binding agent in a furnace.

The non-hydrated hydraulic material may also be applied to the substrate by a PVD or CVD process.

In a fourth optional step, the surface layer is compacted either by application of a high pressure, preferably isostatic, e.g. by using cold isostatic pressing (CIP) or hot isostatic pressing (HIP). The coating may also be compacted by passing a laser beam across the surface. Ideally the degree of compaction of the layer is increased between 30 and 80%, reducing the porosity to 30-45 vol %.

A fifth final step comprises hydration of the ceramic layer by exposing it to water or water vapour, at a selected temperature. Hydration accelerators (preferably LiCl) or retarders may be added to the hydration water. The hardening of the material may be performed in the temperature range from approximately 10 to 100° C., in water or vapour. Preferably, the hardening is performed in the range 20 to 70° C. Temperatures above 100° C. may also be used for vapour. Temperatures above 30° C. leads to fast formation of stable hydrate phases. If shorter hardening times and more complete hydration are desired, the more elevated temperatures may be used. The use of autoclavation is a technique of achieving the preferred hardening conditions in terms of temperature and humidity.

In an alternative embodiment of the invention the calcium aluminate or calcium silicate coating material is used as binder phase (carrier) for other biocompatible and/or osseo-compatible materials, whereby unique combinatory properties can be achieved. The method comprises adding particles or powder of one or more biocompatible materials to the hydraulic phase. The biocompatible materials suitably include different types of calcium carbonates, calcium phosphates and hydroxyapatites. Addition of other apatites, such as fluorapatite or carbonates-apatites, is also relevant to the invention.

Due to the low temperatures involved in the deposition process, these materials, being temperature-sensitive, can be carried by the calcium aluminate coating, and their phase composition be preserved.

Preferably, powders of inert filler materials or biocompatible materials are added to the hydraulic ceramic powder during its preparation. Such a calcium aluminate coating with improved biocompability may provide implants with osseo-compatible properties in applications where coatings of pure hydroxyapatite or the like are too weak.

The characteristics of the process and the coating material make it possible to deposit coatings on devices that are sensitive to high temperatures due to a low melting point, temperature expansion, hardening procedures and the like.

### EXAMPLE

Titanium substrates were pretreated by sand blasting with hard grit. In a first step, the surface was blasted with 100-120 mesh aluminum oxide grit to a surface roughness of Rₐ between 0.6 and 0.7 μm. In an additional optional step, not necessary for the process, a secondary blasting of the titanium surface with calcium aluminate (CA) was performed with grit of about 22 μm.

A calcium aluminate powder from Lafarge Aluminates, Ternal White® was selected. This is a calcium aluminate with a ratio of Al₂O₃ and CaO of about 70/30. However, any other similar calcium aluminate powder would lead to similar results. The grain size of this powder was reduced my ball milling. The milling reduced the size of 90% of the grains to less than 10 μm. The milling was performed with a rotating cylindrical plastic container using 10 mm in diameter silicon nitride spheres as milling medium. The milling liquid was isopropanol. The total milling time was 72 hrs.

After milling, the milling bodies were removed by sieving and the alcohol was evaporated. Thereafter the milled powder was burnt at 400° C. for 4 hours, to remove any residual water and organic contamination.

The powder was applied to the surface of the pretreated titanium substrate either as layers of about 300-500 microns by dipping, or as layers of about 20-50 microns by spraying. To apply the layers, a slurry of milled calcium aluminate powder in isopropanol was prepared. About three quarters of alcohol was mixed with about one quarter of powder, by volume. After deposition, the alcohol was evaporated. The dried powder remained as a weakly bonded layer, covering the entire substrate.

For a selection of samples the layers were compacted together with their substrate with cold isostatic pressing at pressures between 200-300 MPa. Following the standard procedure for cold isostatic pressing, the samples were put in polymer bags, and the pressure medium was oil in a hydraulic system. The open porosity of the layers was between 30 and 40% after compaction.

Thereafter, the samples were hydrated in a closed container with a layer of deionised water at the bottom at 37° C., leading to a saturated water vapour environment, for at least 24 hrs.

The mechanical properties of the layers/coatings were evaluated as Vicker hardness. It was found that the compaction of the layer prior to hydration increased the hardness from about 60-80 to between 130 and 160 on the Vicker hardness scale.

1. Surface coating method comprising the steps:
preparing a powder mixture based on a non-hydrated hydraulic ceramic powder binder phase,
pretreating a substrate surface, to increase the adhesion between the substrate and the ceramic coating,
applying at least one layer of the non-hydrated powder mixture on the substrate, and
hydrating the powder layer/layers by addition of a water-based solution to harden the ceramic coating.

2. Surface coating method according to claim 1, wherein the step of preparing a powder mixture comprises adding particles or powder of one or more non-hydraulic filler materials.

3. Surface coating method according to claim 2, wherein the non-hydraulic filler powder comprises a ternary oxide of perovskite structure according to the formula ABO₃, where O is oxygen and A and B are metals, or any mixture of such ternary oxides.

4. Surface coating method according to claim 3, wherein the ternary oxide is calcium titanate.

5. Surface coating method according to claim 1, wherein the step of preparing a powder mixture comprises adding particles or powder of one or more biocompatible materials.

6. Surface coating method according to claim 5, wherein the biocompatible material is selected from the group consisting of calcium carbonate, calcium phosphate, apatite, fluoroapatite, carbonates-apatites, and hydroxyapatite.

7. Surface coating method according to claim 1, wherein the step of preparing a powder mixture includes reducing the powder grain size such that it is to more than 50 vol.%, preferably to more than 80 vol.% and most preferably more than 90 vol.% in the range 0.5-20 microns.

8. Surface coating method according to claim 1, wherein the pretreatment of the substrate surface is performed to a surface roughness in the range of Rₐ 0.1 to 10.0 μm before applying of the powder mix.

9. Surface coating method according to claim 1, wherein the pretreatment of the substrate is performed by blasting with hard particles.

10. Surface coating method according to claim 1, comprising the step of embedding calcium aluminate fragments in the substrate surface.

11. Surface coating method according to claim 11, wherein the embedding is performed by blasting the surface with calcium aluminate fragments or powder.

12. Surface coating method according to claim 1, wherein the non-hydrated ceramic layer is applied by a thermal spray technique, PVD or CVD deposition techniques, or applied as a tape prepared by tape casting.

13. Surface coating method according to claim 1, wherein the applied non-hydrated ceramic powder layer/layers are compacted, for example by using cold isostatic pressing (CIP), hot isostatic pressing (HIP), or by passing a laser beam across the surface, prior to the final hydration.

14. Surface coating method according to claim 15, wherein the degree of compaction of the powder layer is increased between 30 and 80% and the porosity reduced to 30-45 vol.%.

15. Surface coating method according to claim 1, wherein the step of hardening the ceramic coating comprises addition of a component which accelerates or retards the hardening process.

16. Surface coating method according to claim 1, wherein the step of hardening is performed in water or in water vapour.

17. Surface coating method according to claim 1, wherein the step of hardening comprises controlling the temperature to be in the range of 10° C. to 200° C., preferably in the range 20° C. to 70° C.

18. Surface coating method according to claim 1, wherein the applied coating has a thickness in the order of 0.1-500 μm, and preferably is less than 50 μm.

19. Surface coating method according to claim 1, wherein the non-hydrated hydraulic ceramic powder comprises calcium aluminate or calcium silicate.

20. Surface coating method according to claim 1, wherein the substrate comprises Ti or alloys thereof, stainless steel, Co—Cr alloys, another biocompatible metal, polymeric or ceramic material, or any combination thereof.

21. Method of producing a surface coated biocompatible device, comprising the steps:
preparing a powder mixture based on a non-hydrated hydraulic ceramic powder binder phase,
pretreating a medical device substrate surface, to increase the adhesion between the substrate and the ceramic coating,
applying at least one layer of the non-hydrated powder mixture on the medical device surface, and
hydrating the powder layer/layers by addition of a water-based solution to harden the ceramic coating.

22. Method of producing a surface coated biocompatible device according to claim 21, wherein the non-hydrated hydraulic ceramic powder comprises calcium aluminate or calcium silicate.

23. Method of producing a surface coated biocompatible device according to claim 21, wherein the step of preparing a powder mixture comprises adding particles or powder of one or more non-hydraulic filler materials.

24. Method of producing a surface coated biocompatible device according to claim 23, wherein the non-hydraulic filler powder comprises a ternary oxide of perovskite structure according to the formula ABO₃, where O is oxygen and A and B are metals, or any mixture of such ternary oxides.

25. Method of producing a surface coated biocompatible device according to claim 24, wherein the ternary oxide is calcium titanate.

26. Biocompatible surface coating, wherein a binder phase in the coating substantially is comprised of calcium aluminate hydrate, or calcium silicate hydrate.

27. Biocompatible surface coating according to claim 26, further comprising a ternary oxide of perovskite structure described by the formula ABO₃, where O is oxygen and A and B are metals, or any mixture of such.

28. Biocompatible surface coating according to claim 27, wherein the ternary oxide is calcium titanate.

29. Biocompatible surface coating according to claim 26, further comprising particles or fragments of one or more biocompatible materials selected from the group consisting of calcium carbonate, calcium phosphate, apatite, fluorapatite, carbonates-apatites, and hydroxyapatite.

30. Biocompatible surface coating according to claim 26, having a thickness in the order of 0.1-500 μm, and preferably less than 50 μm.

31. Surface coated device, comprising a substrate and a surface coating covering at least a section of the substrate surface, wherein the surface coating is a biocompatible surface coating comprising a binder phase substantially comprised of calcium aluminate hydrate, or calcium silicate hydrate.

32. Surface coated device according to claim 31, the surface coating of which comprises one or more non-hydraulic filler materials.

33. Surface coated device according to claim 32, the non-hydraulic filler material being a ternary oxide of perovskite structure described by the formula ABO₃, where O is oxygen and A and B are metals, or any mixture of such.

34. Surface coated device according to claim 33, wherein the ternary oxide is calcium titanate.

35. Surface coated device according to claim 31, further comprising particles or fragments of one or more biocompatible materials selected from the group consisting of calcium carbonate, calcium phosphate, apatite, fluorapatite, carbonates-apatites, and hydroxyapatite.

36. Surface coated device according to claim 31, having a thickness in the order of 0.1-500 μm, and preferably less than 50 μm.

37. Surface coated device according to claim 31, wherein the substrate is Ti or alloys thereof, stainless steel, Co—Cr alloys, another biocompatible metal, polymeric or ceramic material, or any combination thereof.

38. Surface coated device according to claim 31, which is a medical device, medical device for implantation, artificial orthopedic device, spinal implant, joint implant, attachment element, bone nail, bone screw, or a bone reinforcement plate.
